(19) United States
(12) Patent Application Publication
(10) Pub. No.: US 2022/0380828 A1
(43) Pub. Date: Dec. 1, 2022

(54) MICROBIAL DETECTION PLATFORM

(71) Applicant: NUCLEASE PROBE TECHNOLOGIES, INC., Lowell, MA (US)

(72) Inventors: James O. MCNAMARA, Lowell, MA (US); Nodar MAKHARASHVILI, Lowell, MA (US)

(21) Appl. No.: 17/773,667

(22) PCT Filed: Nov. 5, 2020
(86) PCT No.: PCT/US20/59016

§ 371 Date: May 2, 2022

Related U.S. Application Data
(60) Provisional application No. 62/930,616, filed on Nov. 5, 2019.

(51) Int. Cl. C12Q 1/08; C12Q 1/04

(52) U.S. Cl. CPC C12Q 1/08; C12Q 1/045

(57) ABSTRACT

The present invention is related to a nuclease-activated culture substrate, a method of rapidly detecting an antibiotic-resistant microorganism using the nuclease-activated culture substrate, and kits including the nuclease-activated culture substrate.

### TECHNICAL FIELD

The present invention in various aspects and embodiments relates to microbial detection, including detection of drug-resistant pathogens.

### BACKGROUND

Antibiotic-resistant bacteria are rapidly emerging worldwide and endangering the efficacy of antibiotics that have transformed medicine and saved millions of lives (Golkar et al., J. Infect. Dev. Ctries. 2014; 8(2):129-136; Gould et al., Virulence. 2013; 4(2):185-191; Wright, Can. J. Microbiol. 2014; 60(3):147-154; Sengupta et al., Front. Microbiol. 2013; 4:47). The antibiotic-resistance crisis has been attributed to the overuse and misuse of antibiotics. Antibiotic-resistant bacteria are placing a substantial burden on the U.S. health care system and the Center for Disease Control and Prevention (CDC) has classified a number of bacteria as presenting urgent, serious, and concerning threats.

Infections by antibiotic-resistant microorganisms are often difficult to treat because the weakest link in the overall treatment strategy is the screening, detection, and/or identification of the antibiotic-resistant microorganism. Known tests used for identification of such microorganisms vary in terms of complexity, completion times, and in the level of precision. The time taken for detection and identification often takes from 18 to 24 hours and even longer in some cases. When antimicrobial susceptibility tests are used to aid in diagnosis of the microorganism, bacterial identification is most likely to take around 48 hours after initial sample reception. Such time delays can hamper patient treatment and have harmful implications in terms of infection control.

Usually, detection methods for pathogens involve culture in liquid or on solid media. These techniques, including use of a chromogenic cefoxitin-based agar medium, typically detect Methicillin resistant and Methicillin sensitive S. aureus within 20 to 48 hours. Rapid MRSA/MSSA detection is possible using PCR and hybridization assays, however, these assays require sophisticated laboratory equipment and well-trained professionals. Speed of diagnosis, detection, and identification is critical in the management of patients and in the control of bacterial infection.

Accordingly, there is a need in the field for alternative microbial detection platforms that provide rapid and accurate detection of pathogenic bacteria, and which can have reduced labor and material costs, and/or which require fewer biochemical and/or serological confirmation tests in order to confirm the presence or absence of pathogenic bacteria.

### SUMMARY OF THE INVENTION

The present invention provides nuclease-activatable culture substrates and methods for rapidly detecting microorganisms of interest using such nuclease-activatable culture substrates. In various embodiments, the invention provides methods for detecting antibiotic-resistant bacteria.

Conventional methods for detecting antibiotic-resistant microorganisms involve processes requiring at least 24 hours or even days of incubation of the microorganisms. Moreover, the conventional methods often require biochemical or serological confirmation tests in order to confirm the identity of the microorganism. In various embodiments, the methods disclosed herein have the advantage of permitting one to detect and identify antibiotic-resistant microorganisms by using a simple and cost-effective nuclease-activatable culture substrate. For example, the methods disclosed herein often take less than 8 hours, or in some embodiments less than 6 hours, and can be implemented without the need for sophisticated or expensive instruments.

In one aspect, the present invention provides a method for detecting microorganisms of interest in a sample. This method includes providing a culture substrate to support growth of the microorganisms of interest, which can be antibiotic-resistant bacterial species. The culture substrate includes one or more antimicrobial agents (e.g., antibiotics or antifungal agents) and a nucleic acid probe that emits a fluorescent signal upon endonuclease cleavage. The method also includes contacting the culture substrate with the sample, and incubating the culture substrate for a period of time to allow growth of the microorganisms of interest, and detecting the presence, absence, or level of fluorescent signal in the culture substrate or a portion thereof. In this method, detection of a fluorescent signal that is greater than that of a control or baseline level indicates the presence of microorganisms of interest. The control or baseline fluorescent signal can be, e.g., measured from a) a portion of the culture substrate where the sample was not applied; or b) a culture substrate that does not include the nucleic acid probe.

Another aspect of the present invention is related to a nuclease-activatable culture substrate for detecting microorganisms of interest, including in some embodiments, antibiotic-resistant bacteria. The culture substrate includes an agar-containing growth medium; one or more antimicrobial agent (e.g., antibiotics or antifungal agents); and a nucleic acid probe that emits a fluorescent signal upon endonuclease cleavage, wherein the nuclease-activatable culture substrate is capable of supporting growth of the antimicrobial agent-resistant microorganisms.

The nucleic acid or oligonucleotide probes are 2-30 nucleotides in length and include at least one endonuclease cleavage site; at least one fluorescence quencher linked to the oligonucleotide; and at least one fluorophore linked to the oligonucleotide. In some embodiments, the fluorophore and fluorescence quencher flank the endonuclease cleavage site. The endonuclease cleavage site has specificity for an endonuclease produced by the microorganism of interest. Endonuclease cleavage of the nucleic acid probe separates the fluorophore from the fluorescence quencher and allows generation of the fluorescent signal. Detection of this fluorescent signal indicates the presence of microorganisms of interest in the sample. The nucleic acid probes may include chemically modified nucleotides and, in some instances, the chemical modification provides the nucleic acid probe resistance against mammalian endonucleases or other nucleases that may be present in the sample. The chemical modification of the nucleic acid probe can be, e.g., 2′-O-methyl or 2′-fluoro modifications.

The present disclosure provides for rapid detection of microorganisms of interest in a sample, including antibiotic-resistant bacteria. In some embodiments, the presence, absence, or level of the fluorescent signal is detected within about 15 hours of incubation, within about 12 hours of incubation, within about 10 hours, within about 8 hours, or within about 6 hours of incubation. In some embodiments, the presence, absence, or level of the fluorescent signal is detected within about 4 hours of incubation or about 2 hours of incubation. In these embodiments, the invention allows for effective screening of important pathogens, including Staphylococcus aureus, Staphylococcus pyogenes, and E. coli. Further, in various embodiments, the fluorescent signal provides for detection of methicillin resistant S. aureus (MRSA), or carbapenem resistant Enterobacteriaceae (CRE).

The culture substrates and methods described herein can be used for detection of bacterial contamination in research laboratories, food and water testing, veterinary diagnostic applications, medical diagnostic applications and medical diagnostic imaging.

Other aspects and embodiments of the invention will be apparent from the following detailed description.

### BRIEF DESCRIPTION OF THE DRAWINGS

FIGS. 1A-C show S. aureus detection with S. aureus-specific NPT probe. SASelect (of Bio-Rad) agar plates and agar plates embedded with AttoPoly T 4mer S. aureus-specific probe were streaked with <1000 cells of E. coli (ATCC 25922), S. aureus (ATCC 29213), and S. lugdunensis (ATCC 700328)) incubated at 37° C. FIG. 1A shows a schematic representation of plates. FIG. 1B shows SASelect agar plates. FIG. 1C shows NPT probe-embedded agar plates (NucAP™). The times are intervals between inoculation and image acquisition.

FIG. 2 shows rapid and specific detection of S. aureus on agar growth media with a nuclease-responsive probe. 96-well plates with TSA-based media that includes 2 μM AttoPolyT 4mer S. aureus-specific probe. The indicated cell numbers (approximated via OD) were added to wells and the plate was incubated at 37° C. Averages of triplicates after background subtraction are shown in upper panel. Fluorescence of individual wells at indicated time points for the 1 cell/well dilutions are shown in middle panel. Note the elevated levels in two wells of S. aureus at the later time points (solid arrows in middle and lower panels). Fluorescence was measured with a Bio-Tek plate-reader (upper and middle panels) or IORodeo/iPhone camera (lower panel).

FIG. 3 shows time-course of Methicillin-susceptible (MSSA-ATCC 29213) and Methicillin-resistant (MRSA-ATCC BAA-1707) S. aureus detection. No probe or the indicated concentrations of S. aureus-specific AttoPoly T 4mer probe was embedded in TS-agar. The plates were streaked with 10,000 cells (approximated via OD) of indicated strains and incubated at 37° C. Images were taken every 1 hr; selected images are displayed. The time labels indicate the intervals between inoculation and image acquisition. Images were acquired with an IORodeo LED illumination source and an iPhone camera.

FIG. 4 shows time-course of Methicillin-susceptible (MSSA-ATCC 29213) and Methicillin-resistant (MRSA-ATCC BAA-1707) S. aureus detection and identification in the presence or absence of 4 μg/mL Cefoxitin. The S. aureus-specific probe AttoPoly T 4mer was embedded at 1 μM concentration in TS-agar. The plates were streaked with ˜10,000 cells (approximated via OD) of indicated strains and incubated at 37° C. Images were taken every hour; selected images are displayed. The time labels indicate the intervals between inoculation and image acquisition. Images were acquired with an IORodeo LED illumination source and an iPhone camera.

FIG. 5 shows rapid MRSA and MSSA detection with S. aureus-specific probe embedded in agar with/without cefoxitin. 96-well plates were filled with TSA-based media that includes 1 μM of the S. aureus-specific AttoPolyT 4mer probe. The indicated bacterial cell numbers (approximated with OD) of MRSA and MSSA were added and the plate was incubated at 37° C. MRSA (ATCC BAA1707) can be distinguished from MSSA (ATCC 29213) in 8-10 hours. Values shown are averages of duplicates. Fluorescence was measured with a Bio-Tek plate-reader.

FIG. 6 is related to rapid and specific detection of E. coli with fluorogenic agar. 96-well plates were filled with a TSA-based media that includes 0.5 μM of the Self-Hyb ATTO E. coli-responsive probe. The indicated cell numbers (approximated with OD) of E. coli (ATCC 25922), S. aureus (ATCC 29213), and S. pyogenes (ATCC 12344) were added and the plate was incubated at 37° C. E. coli can be specifically detected as early as 8 hours. Values shown are averaged triplicates; fluorescence of the 1 hour time-point (representing background) was subtracted from each. Fluorescence levels were measured with a Bio-Tek plate-reader.

FIG. 7 shows S. pyogenes (ATCC 12344) detection with a strain-specific NPT probe. Agar plates were embedded with 0.5 μM Self-Hyb ATTO probe and streaked with 10,000 cells (approximated via OD) of indicated strain and incubated at 37° C. S. pyogenes can be detected in 19 hours, even in the absence of visible bacterial colonies, which appear after 31 hours.

FIG. 8 is related to rapid detection of S. pyogenes on selective fluorogenic agar. 96-well plates were filled with a TSA-based media that includes 0.5 μM of the Self-Hyb ATTO S. pyogenes-responsive probe. The indicated cell numbers (approximated with OD) of S. pyogenes (ATCC 12344) were added and the plate was incubated at 37° C. Low concentrations of S. pyogenes can be detected in the presence of colistin (2 μg/ml), which eliminates growth of off-target gram-negative species, within 10 hours. Fluorescence levels were measured with a Bio-Tek plate-reader. Values shown are averaged triplicates; error bars (S.D.) were omitted from the no-colistin 1 cell/well 20 hr value due to the high error in this triplicate (likely due to a well lacking bacteria in this triplicate). Note that the absence of signal in the colistin-containing samples with ˜1 CFU/well indicates that these wells likely had no bacterial cells present.

### DETAILED DESCRIPTION

The present invention is related to a nuclease-activatable culture substrate and associated methods for identification/detection of a microorganism of interest where the culture substrate includes a nucleic acid probe that i) includes a site that is cleavable by an endonuclease, ii) may have one or more chemical modifications to enhance the specificity of the probe, and iii) includes a fluorophore and a fluorescence quencher. In some embodiments, the target microorganism is identified due to cleavage of the nucleic acid probe by the target microorganism's nucleases (e.g., ribonuclease or an endonuclease). Upon cleavage of the nucleic acid probe, the fluorophore diffuses away from the fluorescence quencher and a fluorescent signal is generated. This fluorescent signal can be detected and indicates presence of the target microorganism.

In some embodiments, the microorganism of interest is an antibiotic-resistant microorganism and the culture substrate includes an antimicrobial agent (e.g., antibiotic or antifungal agent) which allows growth of only antibiotic-resistant microorganisms as well as the nucleic acid probe described above. In embodiments where the culture substrate includes both the antimicrobial agent and the nucleic acid probe, the detection of the antibiotic-resistant microorganism is performed in a reduced amount of time as compared to a culture substrate that does not include the nucleic acid probe. In some embodiments, inclusion of both the antibiotic and the nucleic acid probe allows for more precise selection/identification of the microorganism of interest.

In one aspect, the present invention provides a method for detecting microorganisms of interest in a sample. This method includes providing a culture substrate to support growth of the microorganisms of interest. The culture substrate includes one or more antimicrobial agents and a nucleic acid probe that emits a fluorescent signal upon endonuclease cleavage. The method also includes contacting the culture substrate with the sample, and incubating the culture substrate for a period of time to allow growth of microorganisms of interest and detecting the presence, absence, or level of fluorescent signal in the culture substrate or a portion thereof. In this method, detection of a fluorescent signal that is greater than that of a control or baseline level indicates the presence of microorganisms of interest, which in some embodiments are antibiotic-resistant bacteria. The control or baseline fluorescent signal can be, e.g., measured from a) a portion of the culture substrate where the sample was not applied; or b) a culture substrate that does not include the nucleic acid probe.

Another aspect of the present invention provides a nuclease-activatable culture substrate for detecting microorganisms of interest. The culture substrate includes an agar-containing growth medium; one or more antimicrobial agents (e.g., antibiotics or antifungal agents); and a nucleic acid probe that emits a fluorescent signal upon endonuclease cleavage, wherein the nuclease-activatable culture substrate is capable of supporting growth of the microorganisms of interest.

In some embodiments, the method of the present invention is based on detection of a fluorescent signal generated when the nucleic acid probe is cleaved by the target organism's endonuclease. The nucleic acid probe may include one or more sites for a target organism's nuclease, a fluorophore, and a fluorescence quencher and may be designed such that it generates a fluorescent signal when the target organism's nuclease cleaves the nucleic acid probe. In some embodiments, the nucleic acid probes are not cleaved by mammalian nucleases that may be present in the sample, but are cleaved by nucleases produced by target microorganisms, including pathogenic bacteria, such as, Staphylococcus aureus, Staphylococcus pyogenes, and E. coli. The probes can thus be used to detect the presence of target microorganisms in biological samples such as blood or serum, urine, tissue swabs, surfaces, food, and the likes.

In some embodiments, the culture substrate includes a nucleic acid probe that emits a fluorescent signal upon cleavage by an endonuclease. This cleavage of the nucleic acid by a target organism's endonuclease provides a specific, sensitive, and efficient means of detecting the target organism in a sample. Further, these methods provide a shortened time period for detecting a target microorganism. Generation of the fluorescent signal from the culture substrate indicates the presence, absence, or quantity of target microorganism in a sample. In some embodiments, the presence, absence, or level of fluorescent signal is detected within about 2 to about 18 hours of incubation. In some embodiments, the presence, absence, or level of fluorescent signal is detected within about 12 hours of incubation, within about 10 hours, within about 8 hours, or within about 6 hours of incubation. In some embodiments, the presence, absence, or level of fluorescent signal is detected within about 4 hours of incubation or about 2 hours of incubation.

In some embodiments, the culture substrate includes an agar-containing growth medium, which can be inoculated with a sample. In other embodiments, the culture substrate includes solid matrices other than agar, including various polysaccharide gels, e.g., carrageenan.

In some embodiments, the fluorescent signal generated by the nucleic acid probe is detected using a fluorometer or other means of detecting fluorescence available in the art. In some embodiments, the target microorganism is detected by visualizing the colonies formed on a culture substrate using fluorescence. In some embodiments, the colonies are quantified using fluorescence as a means for diagnosing bacterial infection.

The oligonucleotide is a substrate for nuclease (e.g., ribonuclease) enzyme and includes i) one or more nuclease-cleavable bases, e.g., RNA bases, some or all of which function as scissile linkages, ii) a fluorescence-reporter group and a fluorescence-quencher group (in a combination and proximity that permits visual FRET-based fluorescence quenching detection methods), and iii) may optionally contain RNase-resistant modified RNA bases, nuclease-resistant DNA bases, or unmodified DNA bases. Synthetic oligonucleotide RNA-DNA chimeras wherein the internal RNA bonds function as a scissile linkage are described, e.g., in U.S. Pat. Nos. 6,773,885 and 7,803,536, which are hereby incorporated by reference in their entireties. The fluorescence-reporter group and the fluorescence-quencher group are separated by at least one RNAse-cleavable residue, e.g., RNA base. Such residues serve as a cleavage domain for ribonucleases.

In some embodiments, the fluorescent nucleic acid probe includes an oligonucleotide of 2-30 nucleotides in length. In embodiments, the oligonucleotide comprises one or more pyrimidines and at least one of the pyrimidines of the oligonucleotide is chemically modified. In other embodiments, the oligonucleotide comprises one or more purines and at least one of the purines of the oligonucleotide is chemically modified. In some embodiments, one or more pyrimidines are 2′-O-methyl modified or 2′-fluoro modified and, in other embodiments, one or more purines are 2′-O-methyl modified or 2′-fluoro modified.

In certain embodiments, the oligonucleotide is single-stranded or double-stranded. In some embodiments, the nucleic acid probe includes two oligonucleotides that are completely self-complementary yielding a double-stranded nucleic acid. In still other embodiments, the oligonucleotide is a single, self-hybridizing oligonucleotide. In certain embodiments, the oligonucleotide is composed of modified ribonucleotides. The term “modified” encompasses nucleotides with a covalently modified base, sugar, or phosphate group. For example, modified nucleotides include nucleotides having sugars which are covalently attached to low molecular weight organic groups at positions other than at the 3′ position or the 5′ position. Thus, modified nucleotides may also include 2′ substituted sugars such as 2′-O-methyl-; 2-O-alkyl; 2-O-allyl; 2′-S-alkyl; 2′-S-allyl; 2′-fluoro-; 2′-halo or 2-azido-ribose, carbocyclic sugar analogues α-anomeric sugars; epimeric sugars such as arabinose, xyloses or lyxoses, pyranose sugars, furanose sugars, and sedoheptulose. In certain embodiments, the oligonucleotide includes, but is not limited to, 2′-O-methyl RNA, 2′-methoxyethoxy RNA, 2′-O-allyl RNA, 2′-O-pentyl RNA, and 2′-O-butyl RNA. In certain embodiments, the oligonucleotide is an RNA-2′-O-methyl oligonucleotide having the general structure 5′ r-NnN-q 3′, where ‘N’ represents from about one to five 2′-modified ribonucleotide residues, ‘n’ represents one to ten unmodified ribonucleotide residues, ‘r’ represents a fluorescence reporter group, and ‘q’ represents a fluorescence quencher group. The 5′- and 3′-position of reporter and quencher are interchangeable. In one embodiment, the fluorescence reporter group and the fluorescence quencher group are positioned at or near opposing ends of the oligonucleotide, however, it is not required that the reporter and quencher groups be end modifications as long as cleavage of the oligonucleotide by the nuclease results if a fluorescent signal. In one embodiment, the fluorescence reporter group and the fluorescence quencher group may be positioned internally so long as a nuclease scissile linkage lies between reporter and quencher.

Modified nucleotides are known in the art and include, by example and not by way of limitation, alkylated purines and/or pyrimidines; acylated purines and/or pyrimidines; or other heterocycles. These classes of pyrimidines and purines are known in the art and include, pseudoisocytosine; N4, N4-ethanocytosine; 8-hydroxy-N6-methyladenine; 4-acetylcytosine, 5-(carboxyhydroxylmethyl) uracil; 5-fluorouracil; 5-bromouracil; 5-carboxymethylaminomethyl-2-thiouracil; 5-carboxymethylaminomethyl uracil; dihydrouracil; inosine; N6-isopentyl-adenine; 1-methyladenine; 1-methylpseudouracil; 1-methylguanine; 2,2-dimethylguanine; 2-methyladenine; 2-methylguanine; 3-methylcytosine; 5-methylcytosine; N6-methyladenine; 7-methylguanine; 5-methylaminomethyl uracil; 5-methoxy amino methyl-2-thiouracil; β-D-mannosylqueosine; 5-methoxycarbonylmethyluracil; 5-methoxyuracil; 2-methylthio-N6-isopentenyladenine; uracil-5-oxyacetic acid methyl ester; psueouracil; 2-thiocytosine; 5-methyl-2 thiouracil, 2-thiouracil; 4-thiouracil; 5-methyluracil; N-uracil-5-oxyacetic acid methylester; uracil 5-oxyacetic acid; queosine; 2-thicytosine; 5-propyluracil; 5-propylcytosine; 5-ethyluracil; 5-ethylcytosine; 5-butyluracil; 5-pentyluracil; 5-pentylcytosine; and 2,6, -diaminopurine; methylpsuedouracil; 1-methylguanine; 1-methylcytosine.

The oligonucleotides of the disclosure may be synthesized using conventional phosphodiester linked nucleotides and synthesized using standard solid or solution phase synthesis techniques which are known in the art. Linkages between nucleotides may use alternative linking molecules. For example, linking groups of the formula P(O)S, (thioate); P(S)S, (dithioate); P(O)NR′2; P(O)R; P(O)OR6; CO; or CONR′2 wherein R is H (or a salt) or alkyl (1-12C) and R6 is alkyl (1-9C) is joined to adjacent nucleotides through —O— or —S—.

In certain embodiments of the present disclosure, the oligonucleotides have additional modifications, such as 2′ O-methyl modification of the pyrimidines. In other embodiments, all of the nucleotides in the oligonucleotides are 2′ O-methyl modified. Alternatively, the pyrimidines, or all the nucleotides, may be modified with 2′ fluoro (both pyrimidines and purines).

The oligonucleotides are short, such as between 2-30 nucleotides in length (or any value in between). In certain embodiments, that oligonucleotide is between 4-15 nucleotides in length. In certain embodiments, that oligonucleotide is between 4-10 nucleotides in length. In certain embodiments, the oligonucleotide comprises 0-50% purines (or any value in between). In certain embodiments the oligonucleotide comprises 100% pyrimidines. In some embodiments, the oligonucleotide comprises both RNA and DNA. The oligonucleotide can also include only DNA or only RNA. In some embodiments, the oligonucleotide includes a poly-deoxythymidine sequence. In some embodiments, the oligonucleotide comprises a poly T sequence that is a 4-mer, 5-mer, 6-mer, 7-mer, 8-mer, 9-mer or a 10-mer.

In other embodiments, the oligonucleotides comprise the one or more of the sequences shown in Table 1 below:

TABLE 1

| Name | Oligo Sequence |
|---|---|
| Self-Hyb | fCfUfAfCfGfUfAfG (SEQ ID NO: 1) |
| ATTO | wherein “f” indicates 2′-fluoro modified nucleotide. |
| 2′-OMe Pyr | mCmUrAmCrGmUrArG (SEQ ID NO: 2) |
| SH | wherein “m” indicates 2′-O-methyl modified |
| | nucleotides, and “r” indicates RNA nucleotides. |
| 2′-OMe SH | mCmUmAmCmGmUmAmG (SEQ ID NO: 3) |
| | wherein “m” indicates 2′-O-methyl modified |
| | nucleotides. |
| DNA-SH | CTACGTAG (SEQ ID NO: 4) |
| | wherein the nucleotides are all DNA nucleotides. |
| SH-F1 5UUU | fUfUfUfAfUfGfCfAfUfAfAfA (SEQ ID NO: 5) |
| | wherein “f” indicates 2′-fluoro modified nucleotide. |
| P2&3 TT | mUTTmUmUmUmUmUmUmUmU (SEQ ID NO: 6) |
| Probe | wherein “m” indicates 2′-O-methyl modified RNA |
| | nucleotides and T indicates deoxythymidine. |

The PolyT probes and the P2&3 TT Probe shown in the Table 1 are sensitive to micrococcal nuclease of S. aureus. The DNA-SH, SH-Fl 5UUU and Self-Hyb ATTO probes shown in Table 1 are sensitive to Endonuclease I, a well-conserved nuclease of the Enterobacteriaceae (which includes, e.g., E. coli, K. pneumoniae). The SH-Fl 5UUU and Self-Hyb ATTO probes shown in Table 1 are sensitive to a nuclease of S. pyogenes. The 2′-OMe Pyr SH probe is expected to be sensitive to nucleases produced by various mycoplasma species such as M. fermentans. The 2′-OMe SH probe is resistant to most nucleases and, therefore, it can provide a highly specific signature of a nuclease that can cleave this probe.

The poly dT probe is responsive to micrococcal nuclease, a secreted nuclease of S. aureus, and enables detection of S. aureus colonies within 8 hours of plating, even from low S. aureus loads. By inclusion of an antibiotic that differentiates MRSA and MSSA (e.g., cefoxitin), MRSA can be detected much more rapidly than conventional plating techniques. Thus, screening of individuals for the presence of MRSA, using, e.g., nasal swabs, can be facilitated in accordance with embodiments of the present invention.

The self-hybridizing probe based on SEQ ID NO:1 (Self-Hyb) is useful for detecting S. pyogenes, the probe can also be responsive to certain gram-negative bacterial species, such as E. coli. By including an antibiotic that prevents the growth of gram-negative species, such as colistin, specific detection of S. pyogenes is enabled. S. pyogenes is the etiologic agent of strep throat (pharyngitis) and other mild infections, and thus the invention in some embodiments enables the screening of these infections (e.g., from throat swabs) with quick turnaround time.

Further, by plating samples on a substrate containing the endonuclease I-responsive probe, the invention provides for discriminating E. coli and other commensal bacteria such as S. aureus and S. pyogenes. These tests can be run in less than 10 hours. By including an antibiotic in the substrate, such as a carbapenem, antibiotic resistant Enterobacteriaceae can be detected (e.g., CRE). Detection of E. coli and drug-resistant E. coli from e.g., the urinary tract, is routinely carried out for diagnosing urinary tract infections. The invention in these embodiments facilitates this process.

Certain combinations of purines and pyrimidines are susceptible to bacterial endonucleases, while resisting mammalian nucleases. Endonucleases are enzymes that cleave the phosphodiester bond within a polynucleotide chain, in contrast to exonucleases, which cleave phosphodiester bonds at the end of a polynucleotide chain. These bacterial nucleases are not sequence-specific like restriction enzymes, which typically require a recognition site and a cleavage pattern. Some endonucleases cleave single-stranded nucleic acid molecules, while others cleave double-stranded nucleic acid molecules.

In some embodiments, the sensitivity and specificity of the probe is dependent on the appropriate buffer conditions. For instance, micrococcal nuclease of S. aureus requires calcium for enzymatic activity. In some embodiments, the nucleases requires magnesium and, in other embodiments, the nuclease does not require a divalent cation. In some embodiments, specificity for nuclease can be achieved with selective media as outlined herein.

The nucleic acid probes can be synthesized using solid-phase phosphoramidite chemistry (see, e.g., U.S. Pat. No. 6,773,885, which is hereby incorporated by reference in its entirety) with automated synthesizers, although other methods of nucleic acid synthesis (e.g., the H-phosphonate method) may be used. Chemical synthesis of nucleic acids allows for the production of various forms of the nucleic acids with modified linkages, chimeric compositions, and nonstandard bases or modifying groups attached in chosen places throughout the nucleic acid's entire length.

The present invention can be used in detection of any target microorganism that can be grown/cultured on the culture substrate, e.g., on a solid medium or a liquid medium. In some embodiments, the target microorganisms that may be detected/identified using the present invention include microorganisms that secrete a nuclease, e.g., an endonuclease. In some embodiments, the target microorganism is resistant to a specific antibiotic. In some embodiments, the antibiotic against the target microorganism is included in the culture substrate and allows for selection and quantification of the antibiotic-resistant microorganism. In some embodiments, the antibiotic is selected from Ceftriaxone, Cefepime, Vabomere, Avycaz, Trimethoprim/Sulfamethoxazole (usually used in combination), Streptomycin, Fosfomycin, Ciprofloxacin, Azithromycin, Amoxicillin; Beta-lactams, such as, Methicillin, Oxacillin, Cefoxitin; Penams, Cephams, Monobactams; Carbapenems, such as, Meropenem, Ertapenem, Imipenem; Cephalosporins and Cephamycins; Beta-lactamase inhibitors, Colistin, Penicillin, Tetracycline, Erythromycin, Gentamicin, Vancomycin, Ceftazidime, Levofloxacin, Linezolid, Daptomycin, and Ceftaroline. In some embodiments, the antibiotic that is included in the culture substrate is identified in the context of the antibiotic-resistant microorganism, e.g., on Center of Disease Control and Food and Drug Administration's Antibiotic Resistance (AR) Isolate Bank.

In some embodiments, the culture substrate includes an antifungal selected from Polyenes, such as, Amphotericin B, Candicidin, Filipin, Hamycin, Natamycin, Nystatin, Rimocidin; Azoles including imidazoles, such as, Bifonazole, Butoconazole, Clotrimazole, Econazole, Fenticonazole, Isoconazole, Ketoconazole, Luliconazole, Miconazole, Omoconazole, Oxiconazole, Sertaconazole, Sulconazole, Tioconazole; Triazoles, such as, Albaconazole, Efinaconazole, Epoxiconazole, Fluconazole, Isavuconazole, Itraconazole, Posaconazole, Propiconazole, Ravuconazole, Terconazole, Voriconazole; Thiazoles, such as, Abafungin; Allylamines, such as, amorolfin, butenafine, naftifine, and terbinafine; Echinocandins, such as, Anidulafungin, Caspofungin, Micafungin.

In some embodiments, the target microorganisms can be a fungal pathogen. For instance, the target microorganism can be a fungal species selected from Candida auris, Aspergillus spp., Candida albicans.

In some embodiments, the target microorganisms can be any pathogenic bacteria. For example, the target microorganism can be a microorganism from The CDC & FDA Antibiotic Resistance (AR) Isolate Bank. The AR Isolate Bank provides curated collections of resistant organisms. Isolates are gathered through CDC's outbreak response and surveillance programs, validated and sequenced for testing, and then curated. The isolates represent samples from healthcare-associated, foodborne, gonorrhea, and community-associated infections.

In some embodiments, the microorganism is selected from Staphylococcus aureus, Staphylococcus epidermidis, Staphylococcus lugdunensis, Staphylococcus saprophyticus, Streptococcus pyogenes, Streptococcus agalactiae, Streptococcus pneumoniae, Streptococcus mutans, Listeria monocytogenes, Corynebacterium diphtheriae, Bordetella pertussis, Clostridium difficile, Clostridium perfringens, Clostridium botulinum, Enterobacter cloacae, Citrobacter freundii, Borrelia burgdorferi, Treponema pallidum, Bacillus anthracis, Bacillus cereus, Enterococcus faecalis, Enterococcus faecium, Pseudomonas aeruginosa, vancomycin-resistant enterococci, Acinetobacter baumannii, Yersinia pestis, Yersinia pseudotuberculosis, Yersinia enterocolitica, Klebsiella pneumoniae, Vibrio cholerae, Salmonella enterica, Salmonella typhi, Escherichia coli, Serratia marcescens, Proteus mirabilis, Enterobacteriaceae, carbapenem-resistant Enterobacteriaceae, Candida auris, Neisseria gonorrhoeae, Neisseria meningitidis, Mycobacterium tuberculosis, Haemophilus influenzae, Legionella pneumophila, Francisella tularensis, Bacteroides fragilis, Brucella abortus, Mycoplasma fermentans, Mycoplasma pneumonia, Mycoplasma genitalium, Mycoplasma bovis, Chlamydia trachomatis, Methicillin-resistant Staphylococcus aureus (MRSA), and/or Methicillin-sensitive Staphylococcus aureus (MSSA).

In some embodiments, the present invention is related to fluorescent probe that is capable of being specifically cleaved by an endonuclease derived from E. coli or by an endonuclease derived from S. aureus. Such probes can be used in the methods or in the nuclease-activatable culture substrates described herein.

The term “biological sample” as used herein means a sample obtained from a subject or a direct clinical sample, for example, a body fluid such as blood, plasma, or urine, or a throat swab, a nasal swab, a throat swab, a stool sample, a saliva sample, a tissue sample, a hair sample, a skin sample, a skin swab, a and bronchial aspirate sample, a bronchial lavage sample, a perianal swab sample, a synovial fluid sample, a cerebrospinal fluid sample, a blood culture sample, a bacterial culture isolate sample, a urine culture sample, and a surgical biopsy sample. A biological sample may also mean a pure culture of bacteria from various environments such as those obtained from a hospital or other location that is prone to infection by bacteria.

In certain embodiments, the nucleic acid molecules of the present invention are operably linked to one or more fluorophores or fluorescent reporter groups. A fluorophore is a molecule that absorbs light (i.e., excites) at a characteristic wavelength and emits light (i.e., fluoresces) at a second lower-energy wavelength. Fluorescence reporter groups that can be incorporated into the nucleic acid molecules of the present invention include, but are not limited to, fluorescein, tetrachlorofluorescein, hexachlorofluorescein, tetramethylrhodamine, rhodamine, cyanine-derivative dyes, Texas Red, Bodipy, and Alexa dyes. Characteristic absorption and emission wavelengths for each of these are well known to those of skill in the art.

The nucleic acid molecule of the present invention is also operably linked to a fluorescence quencher. The fluorescence quencher is a molecule that absorbs or releases energy from an excited fluorophore (i.e., reporter), returning the fluorophore to a lower energy state without fluorescence emission at the wavelength characteristic of that fluorophore. For quenching to occur, reporter and quencher must be in physical proximity, accordingly, the position/location of the fluorophore and the fluorescence quencher on or within the nucleic acid molecule is such that the fluorophore is quenched by the fluorescence quencher when the nucleic acid molecule is intact. When reporter and quencher are separated, e.g., due to cleavage of the nucleic acid, energy absorbed by the reporter is no longer transferred to the quencher and is instead emitted as light at the wavelength characteristic of the reporter. Thus, appearance of a fluorescent signal from the reporter group following removal of quenching is a detectable event and constitutes a “positive signal” indicating presence of a target microorganism in a sample.

Fluorescence quencher groups include molecules that do not emit any fluorescence signal (“dark quenchers”) as well as molecules that are themselves fluorophores (“fluorescent quenchers”). Nucleic acids that employ a “fluorescent quencher” will emit light both in the intact and cleaved states. In the intact state, energy captured by the reporter is transferred to the quencher via FRET and is emitted as light at a wavelength characteristic for the fluorescent quencher. In the cleaved state, energy captured by the reporter is emitted as light at a wavelength characteristic for the reporter. When compositions that employ fluorescent quenchers are used in a FRET assay, detection must be done using a fluorometer. In certain embodiments, nucleic acid probes that employ a “dark quencher” will emit light only in the cleaved state, enabling signal detection to be performed visually (detection may also be done using a fluorometer). Visual detection is rapid, convenient, and does not require the availability of any specialized equipment. It is desirable for the detection assay of the present invention to have visual detection method as an available option. Nucleic acid probes employing a “dark quencher” enable a visual detection of target microorganism while nucleic acid probes employing a “fluorescent quencher” are incompatible with a visual detection assay.

In some embodiments, the nucleic acid probe includes a fluorescence quencher group that does not itself emit a fluorescence signal, i.e. is a “dark quencher.” “Dark quenchers” useful in compositions of the invention include, but are not limited to, dabcyl, QSY.TM.-7, QSY-33 (4′,5-dinitrofluorescein, pipecolic acid amide) and Black-Hole Quenchers™ 1, 2, and 3 (Biosearch Technologies, Novato, Calif.). Assay results (i.e., signal from cleaved nucleic acid probe) can thus be detected visually. Optionally, the fluorescence signal can be detected using a fluorometer or any other device capable of detecting fluorescent light emission in a quantitative or qualitative fashion.

In some embodiments, the nucleic acid probe of the present invention includes at least one fluorophore selected from the fluorophores listed in Table 2.

TABLE 2

| Probe | Excitation (nm) | Emission (nm) |
|---|---|---|
| Hydroxycoumarin | 325 | 386 |
| Alexa fluor | 325 | 442 |
| Aminocoumarin | 350 | 445 |
| Methoxycoumarin | 360 | 410 |
| Cascade Blue | (375); 401 | 423 |
| Pacific Blue | 403 | 455 |
| Pacific Orange | 403 | 551 |
| Lucifer yellow | 425 | 528 |
| Alexa fluor 430 | 430 | 545 |
| NBD | 466 | 539 |
| R-Phycoerythrin (PE) | 480; 565 | 578 |
| PE-Cy5 conjugates | 480; 565; 650 | 670 |
| PE-Cy7 conjugates | 480; 565; 743 | 767 |
| Red 613 | 480; 565 | 613 |
| PerCP | 490 | 675 |
| Cy2 | 490 | 510 |
| TruRed | 490, 675 | 695 |
| FluorX | 494 | 520 |
| Fluorescein | 495 | 519 |
| FAM | 495 | 515 |
| BODIPY-FL | 503 | 512 |
| TET | 526 | 540 |
| Alexa fluor 532 | 530 | 555 |
| HEX | 535 | 555 |
| TRITC | 547 | 572 |
| Cy3 | 550 | 570 |
| TMR | 555 | 575 |
| Alexa fluor 546 | 556 | 573 |
| Alexa fluor 555 | 556 | 573 |
| Tamara | 565 | 580 |
| X-Rhodamine | 570 | 576 |
| Lissamine Rhodamine B | 570 | 590 |
| ROX | 575 | 605 |
| Alexa fluor 568 | 578 | 603 |
| Cy3.5 581 | 581 | 596 |
| Texas Red | 589 | 615 |
| Alexa fluor 594 | 590 | 617 |
| Alexa fluor 633 | 621 | 639 |
| LC red 640 | 625 | 640 |
| Allophycocyanin (APC) | 650 | 660 |
| Alexa fluor 633 | 650 | 688 |
| APC-Cy7 conjugates | 650; 755 | 767 |
| Cy5 | 650 | 670 |
| Alexa fluor 660 | 663 | 690 |
| Cy5.5 | 675 | 694 |
| LC red 705 | 680 | 710 |
| Alexa fluor 680 | 679 | 702 |
| Cy7 | 743 | 770 |
| ATTO488 | 504 | 521 |
| IRDye 800 CW | 774 | 789 |

In some embodiments, the fluorophore has an emission in the near infra-red range. In one embodiment, the fluorophore is ATTO488.

In some embodiments, the nucleic acid probe of the present invention includes at least one fluorescence quencher selected from those listed in Table 3. Additional quenchers that may be used in the nucleic acid probes of the present invention are described in U.S. Pat. No. 7,439,341, which is hereby incorporated by reference in its entirety.

TABLE 3

| Quencher | Absorption Maximum (nm) |
|---|---|
| DDQ-I | 430 |
| Dabcyl | 475 |
| Eclipse | 530 |
| Iowa Black FQ | 532 |
| BHQ-1 | 534 |
| QSY-7 | 571 |
| BHQ-2 | 580 |
| DDQ-II | 630 |
| Iowa Black RQ | 645 |
| QSY-21 | 660 |
| BHQ-3 | 670 |
| IRDye QC-1 | 737 |

In some embodiments, the fluorescence quencher is ZEN fluorescence quencher (“dark quencher”) or Iowa Black RQ Fluorescence quencher (RQSp) (“dark quencher”). Traditional dark quenchers absorb broadly and do not emit light, which allows use of multiple reporter dyes with the same quencher in a single assay. This characteristic allows for expanded options for multiplex assays where one probe can be used to detect multiple target microorganisms. Also, dark quenchers reduce signal cross-talk, simplifying reporter dye detection, making them compatible with a broad range of image analysis instruments. In some embodiments, the fluorophore is a ATTO488 fluorophore, and the fluorescence quencher is ZEN and/or RQSp.

In certain embodiments, the nucleic acid of the probes described in the present invention is linked to the fluorophore and/or quencher by means of a linker. In certain embodiments, an aliphatic or ethylene glycol linker (as are well known to those will skill in the art) may be used for the purpose of attaching the fluorophore or the quencher to the nucleic acid. In certain embodiments, the linker is a phosphodiester linkage. In certain embodiments, the linker is a phosphorothioate linkage. In certain embodiments, other modified linkages between the modifier groups like dyes and quencher and the bases are used in order to make these linkages more stable, thereby limiting degradation to the nucleases.

In certain embodiments, the linker is a binding pair. In certain embodiments, the “binding pair” refers to two molecules which interact with each other through any of a variety of molecular forces including, for example, ionic, covalent, hydrophobic, van der Waals, and hydrogen bonding, so that the pair have the property of binding specifically to each other. Specific binding means that the binding pair members exhibit binding to each other under conditions where they do not bind to another molecule. Examples of binding pairs are biotin-avidin, hormone-receptor, receptor-ligand, enzyme-substrate, IgG-protein A, antigen-antibody, and the like. In certain embodiments, a first member of the binding pair comprises avidin or streptavidin and a second member of the binding pair comprises biotin.

In certain embodiments, the oligonucleotide is linked to the fluorophore and/or quencher by means of a covalent bond. In certain embodiments, the oligonucleotide probe, i.e., an oligonucleotide that is operably linked to a fluorophore and quencher, is also operably linked to a solid substrate. Chemistries that can be used to link the fluorophores and quencher to the oligonucleotide are known in the art, such as disulfide linkages, amino linkages, covalent linkages, etc. In certain embodiments, aliphatic or ethylene glycol linkers that are well known to those with skill in the art can be used. In certain embodiments, phosphodiester, phosphorothioate and/or other modified linkages between the modifier groups like dyes and quencher are used. These linkages provide stability to the probes, thereby limiting degradation to nucleobases.

In some embodiments, the methods of the present invention include the step of illuminating the culture substrate with a light suitable for absorption by the nucleic acid probe and detecting the fluorescent signal emitted by the nucleic acid probe. In some embodiments, the methods of the present invention can be used for detecting the presence, absence, and/or level or quantity of target microorganism present in a sample. For instance, a sample obtained from a subject is allowed to incubate on the culture substrate for a period of time and the fluorescence signal from the culture substrate is detected and/or quantified. If the target microorganism is present in the sample then the culture substrate generates a fluorescent signal due to cleavage of the nucleic acid probe present in the culture substrate. However, if no target microorganism is present in the sample, no fluorescent signal is detected or only a background level of fluorescent signal is detected. In some embodiments, the fluorescent signal is located or is emanating from individual colonies that grow on the culture plate or a portion of the culture plate that is inoculated with a sample containing the target microorganism. In other embodiments, the fluorescent signal is detected from the whole culture, e.g., in instances where a liquid culture is inoculated with the sample and incubated for specific time interval.

In some embodiments, the fluorescent signal obtained from the culture substrate can be used to quantify the amount of target microorganism present in the sample. For example, when a fluorescent signal is detected from a culture substrate its intensity can be measured and compared to a standard curve which compares the intensity of the fluorescent signal with the amount of target microorganisms present in the culture substrate. In some embodiments, the methods of the present invention include comparing the fluorescent signal generated from a culture substrate that is inoculated with the sample containing a target microorganism with a control fluorescent signal. The control fluorescent signal can be, e.g., measured from a) a portion of the culture substrate where the sample was not applied; or b) a culture substrate that does not include the nucleic acid probe.

In some embodiments, the methods of the present invention further include a step of comparing the control fluorescent signal with the fluorescent signal obtained from the culture substrate inoculated with sample. For example, detection of a fluorescent signal that is greater than the control fluorescent signal indicates that the sample contains the target microorganism and detection of a fluorescent signal that is lesser than the control fluorescent signal indicates that the sample does not contain the target microorganism.

In other embodiments, the fluorescent signal can be measured and compared to the control fluorescent signal to quantitate the amount of target microorganism present in the sample using a standard curve. In some embodiments, the methods of the present invention further include the step of enumerating the target microorganism by counting individual colonies of the target microorganism on a culture substrate based on detection of fluorescent signal from the individual colonies.

In some embodiments, the nucleic acid probes described in the present invention emit, e.g., a fluorescence signal. This signal can be detected using methods of fluorometric detection known in the art. In some embodiments, the fluorescent signal is detected using a spectrofluorometer, a microplate reader, a fluorescence microscope, a fluorescence scanner, a flow cytometer, a fluorescence gel imaging device, a combination of a transilluminator, a light filter and a camera, or other fluorescence imaging device. For instance, following incubation of the sample of the culture substrate, fluorescence emission can be detected using a fluorometer. Fluorometric detection equipment includes, but is not limited to, single sample cuvette devices and multi-well plate readers. Use of a multi-well plate format allows for small sample volumes, such as 200 μl or less, and high-throughput robotic processing of many samples at once. This format is used in certain industrial QC settings. The method also provides for the assay to be performed in RNase free cuvettes. Use of fluorometric detection allows for sensitive and quantitative detection.

The present invention is further illustrated by the following non-limiting examples.

### Example 1: Materials and Methods

Preparation of 60 mm Agar Petri Dishes for S. aureus Growth

Tryptic Soy Agar (BD Cat. #236950) was autoclaved according to the manufacturer's instructions and cooled down to 50-60° C. Then, pre-sterilized components, including Tris-HCl (pH 9.0) (adjusted to 50 mM using the following stock solution: Rockland Cat. #MB028-1000) and CaCl₂) (adjusted to 15 mM using the following stock solution: Sigma-Aldrich Cat. #21115-100ML) were added and the resulting mixture was poured into 60 mm Petri dishes (Corning Cat. #430589). 1 μM or 2 μM AttoPoly T 4mer (IDT, custom order) and 4 μg/mL Cefoxitin (Alfa Aesar Cat. #AAJ6689103) were also included where indicated. The agar mixtures were allowed to solidify, then the Petri dishes were sealed with parafilm (Bemis Cat. #999) and stored at 4° C., protected from light.

S. aureus Growth on Petri Dishes and Imaging

S. aureus (ATCC 29213) cultures were grown in Tryptic Soy Broth (BD Cat. #211825) overnight at 37° C. with shaking at 300-400 rpm. Dilutions to 10,000 cells/4 were made and these were streaked on agar Petri dishes with 1 μL calibrated inoculation loops (Fisher Scientific, Fisherbrand Cat. #22363601). The dishes were incubated at 37° C. and imaged using IO-Rodeo fluorescent imager (IO-Rodeo, Large, Blue LED) and an iPhone 8 camera.

Preparation of 96-Well Agar Plates for S. aureus Growth

Tryptic Soy Agar (BD Cat. #236950) was autoclaved according to the manufacturer's instructions and cooled down to 50-60° C. Then, pre-sterilized additives, including Tris*HCl (pH 9.0) (adjusted to 50 mM with the following stock solution: Rockland Cat. #MB028-1000) and CaCl₂) (adjusted to 15 mM with the following stock solution: Sigma-Aldrich Cat. #21115-100ML) were added. 1 μM or 2 μM AttoPoly T 4mer and 4 μg/mL Cefoxitin (Alfa Aesar Cat. #AAJ6689103) were also included where indicated. 90 μL of the agar mixtures were pipetted into the wells of clear-bottom 96-well plates (Thermo Scientific, nunc Cat. #265301) and allowed to solidify. Then the plates were sealed with optically clear film (Applied Biosystems Cat. #4306311) and stored at 4° C., protected from light.

S. aureus Growth in 96-Well Plates and Imaging

S. aureus (ATCC 29213) cultures were grown in Tryptic Soy Broth (BD Cat. #211825) overnight at 37° C. with shaking at 300-400 rpm. Dilutions to 1,000 cells/μL-0.1 cell/μL were made and pipetted (10 μL/well) into the wells of 96-well plates containing the agar mixtures described. Plates were incubated at 37° C. and imaged using an IO-Rodeo fluorescent imager (IO-Rodeo, Large, Blue LED) and an iPhone 8 camera, and fluorescence was also concurrently measured with a Synergy H1 microplate reader (BioTek).

Preparation of 60 mm Agar Petri Dishes for E. coli Growth

Tryptic Soy Agar (BD Cat. #236950) was autoclaved according to the manufacturer's instructions and cooled down to 50-60° C. Then, pre-sterilized components, including Tris*HCl (pH 8.0) (adjusted to 50 mM with the following stock solution: Fisher Scientific Cat. #BP1758-100), MgCl₂ (adjusted to 20 mM with the following stock solution: Invitrogen Cat. #AM9530G), and Triton® X-100 (adjusted to 1% with the following stock: Fisher Scientific Cat. #50-751-7090) were added and the resulting mixture was poured into 60 mm Petri dishes (Corning Cat. #430589). 0.5 μM Self-Hyb ATTO was also included where indicated. The agar mixtures were allowed to solidify, then the Petri dishes were sealed with parafilm (Bemis Cat. #999) and stored at 4° C., protected from light.

E. coli Growth on Petri Dishes and Imaging

E. coli (ATCC 25922) cultures were grown in Tryptic Soy Broth (BD Cat. #211825) overnight at 37° C. with shaking at 300-400 rpm. Dilutions to 10,000 cells/4 were made and streaked on agar Petri dishes with 1 μL calibrated inoculation loops (Fisher Scientific, Fisherbrand Cat. #22363601). The dishes were incubated at 37° C. and imaged using an IO-Rodeo fluorescent imager (IO-Rodeo, Large, Blue LED) and iPhone 8 camera.

Preparation of 96-Well Agar Plates for E. coli Growth

Tryptic Soy Agar (BD Cat. #236950) was autoclaved according to the manufacturer's instructions and cooled down to 50-60° C. Then, pre-sterilized components, including Tris-HCl (pH 8.0) (adjusted to 50 mM with the following stock solution: Fisher Scientific Cat. #BP1758-100), MgCl₂ (adjusted to 20 mM with the following stock solution: Invitrogen Cat. #AM9530G), and Triton® X-100 (adjusted to 1% with the following stock solution: Fisher Scientific Cat. #50-751-7090). 0.5 μM Self-Hyb ATTO was also included where indicated. 90 μL of the agar mixtures were pipetted into the wells of clear-bottom 96-well plates (Thermo Scientific, nunc Cat. #265301) and allowed to solidify, then the plates were sealed with optically clear film (Applied Biosystems Cat. #4306311) and stored at 4° C., protected from light.

E. coli Growth in 96-Well Plates and Imaging

E. coli (ATCC 25922) cultures were grown in Tryptic Soy Broth (BD Cat. #211825) overnight at 37° C. with shaking at 300-400 rpm. Dilutions to 1,000 cells/μL-0.1 cell/μL were made and pipetted (10 μL/well) into the wells of 96-well plates containing the agar mixtures. Plates were incubated at 37° C. and imaged using an IO-Rodeo fluorescent imager (IO-Rodeo, Large, Blue LED) and an iPhone 8 camera, and fluorescence was also measured concurrently with a Synergy H1 microplate reader (BioTek).

Preparation of 60 mm Agar Petri Dishes for S. pyogenes Growth

Tryptic Soy Agar (BD Cat. #236950) was autoclaved according to the manufacturer's instructions and cooled down to 50-60 degrees C. Then, pre-sterilized components including Tris-HCl (pH 8.0) (adjusted to 50 mM with the following stock solution: Fisher Scientific Cat. #BP1758-100) and MgCl₂ (adjusted to 20 mM with the following stock solution: Invitrogen Cat. #AM9530G) were added and the resulting mixture was poured into 60 mm Petri dishes (Corning Cat. #430589). 0.5 μM Self-Hyb ATTO and 2 μg/mL Colistin (Alfa Aesar Cat. #AAJ67415XF) were also included where indicated. The agar mixtures were allowed to solidify. Then the Petri dishes were sealed with parafilm (Bemis Cat. #999) and stored at 4° C., protected from light.

S. pyogenes Growth on Petri Dishes and Imaging

S. pyogenes (ATCC® 12344) cultures were grown in Tryptic Soy Broth (BD Cat. #211825) overnight at 37° C. with shaking at 300-400 rpm. Dilutions to 10,000 cells/4 were made and streaked on agar Petri dishes with 1 μL calibrated inoculation loops (Fisher Scientific, Fisherbrand Cat. #22363601). The dishes were incubated at 37° C. and imaged using an IO-Rodeo fluorescent imager (IO-Rodeo, Large, Blue LED) and an iPhone 8 camera.

Preparation of 96-Well Agar Plates for S. pyogenes Growth

Tryptic Soy Agar (BD Cat. #236950) was autoclaved according to the manufacturer's instructions and cooled down to 50-60° C. Then, pre-sterilized components, including Tris-HCl (pH 8.0) (adjusted to 50 mM with the following stock solution: Fisher Scientific Cat. #BP1758-100) and MgCl₂ (adjusted to 20 mM with the following stock solution: Invitrogen Cat. #AM9530G) were added. 0.5 μM Self-Hyb ATTO and 2 μg/mL Colistin (Alfa Aesar Cat. #AAJ67415XF) were also included where indicated. 90 μL of the agar mixtures were pipetted into the wells of clear-bottom 96-well plates (Thermo Scientific, nunc Cat. #265301) and allowed to solidify. Then the plates were sealed with optically cleared film (Applied Biosystems Cat. #4306311) and stored at 4° C., protected from light.

S. pyogenes Growth in 96-Well Plates and Imaging

S. pyogenes (ATCC® 12344™) cultures were grown in Tryptic Soy Broth (BD Cat. #211825) overnight at 37° C. with shaking at 300-400 rpm. Dilutions to 1,000 cells/μL-0.1 cell/μL were prepared and pipetted (10 μL/well) into the wells of 96-well plates with containing the agar mixtures. Plates were incubated at 37° C. and imaged with an IO-Rodeo fluorescent imager (IO-Rodeo, Large, Blue LED) and an iPhone 8 camera, and fluorescence was also measured concurrently with a Synergy H1 microplate reader (BioTek).

### Fluorogenic Oligonucleotide Probes

Oligonucleotide probes were synthesized and HPLC purified by Integrated DNA Technologies (IDT) of Coralville, IA. The probes consisted of the following: AttoPoly T 4mer: 5′-ATTO488-TTTT-ZEN-RQSp-3′; and Self-Hyb ATTO: 5′-ATTO488-fC-fU-fA-fC-fG-fU-fA-fG-ZEN-RQSp-3′; where ATTO488 is the Atto 488 fluorophore (of ATTO-TEC GmbH, Siegen, Germany), ZEN is the ZEN fluorescence quencher (of IDT), RQSp is the Iowa Black RQ fluorescence quencher (of IDT; attached with a spacer arm). Nucleotides are represented as follows: T is deoxythymidine, fC is 2′-fluoro-modified C, fU is 2′-fluoro modified U, fA is 2′-fluoro-modified A, fG is 2′-fluoro-modified G.

### Example 2: Methods for Detecting Bacteria Using Fluorogenic Agar

Two fluorescence measurement formats were used to detect the growth of target bacterial species on agar growth media. Agar media poured in 60 mm dishes or distributed to the wells of 96-well clear-bottomed plates were placed on an LED transilluminator (IORodeo) that emits blue light. A light filter was placed over the plates or dishes to block the LED-emitted light, but the filter still allows green light to pass, which is captured with an iPhone camera. As the blue light of the LED can efficiently excite the fluorophore included in the oligonucleotide probes (ATTO 488), and this results in green emission, this arrangement provides a means of measuring the target fluorophore, thus indicating nuclease-mediated cleavage of the oligonucleotide probe. The fluorescence of the 96-well plates was also measured with a Bio-Tek fluorescence plate-reader using fluorescence filters tailored for green fluorescence, providing quantitative data.

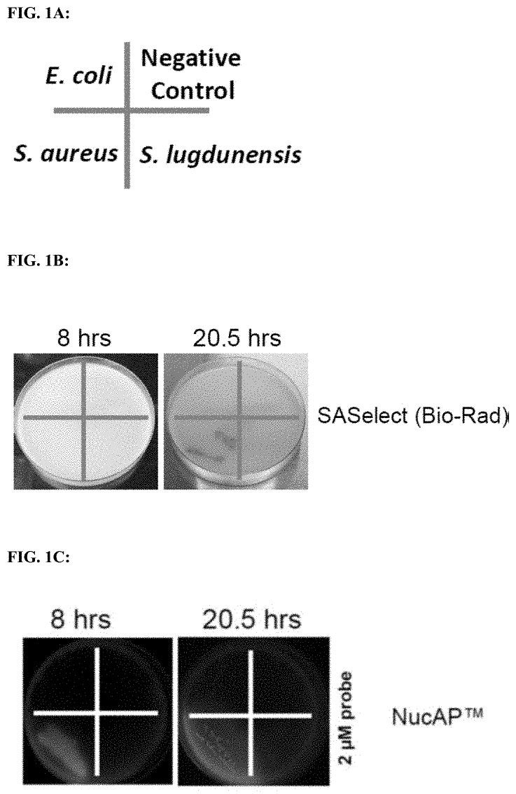

Agar media embedded with a poly-deoxythymidine probe, which is responsive to micrococcal nuclease, a secreted nuclease of S. aureus, enabled robust detection of S. aureus colonies within 8 hours of plating (FIG. 1C, left panel). In contrast, E. coli and S. lugdunensis, which were included to control for specificity, did not produce fluorescence at this early time point, and only produced a low level of fluorescence at later time points, likely attributable to autofluorescence of the bacteria themselves. In addition to elevated fluorescence which co-localized with the S. aureus colonies, at the later 20.5 hour time-point shown, elevated fluorescence was visible in a diffuse pattern throughout the quadrant of the plate in which S. aureus was streaked (FIG. 1C, right panel). The probe-embedded agar, together with fluorescence plate imaging thus enables specific detection of S. aureus as early as 8 hours after plate inoculation. In contrast, a commercial chromogenic agar, SASelect, required several additional hours to detect S. aureus (FIG. 1B).

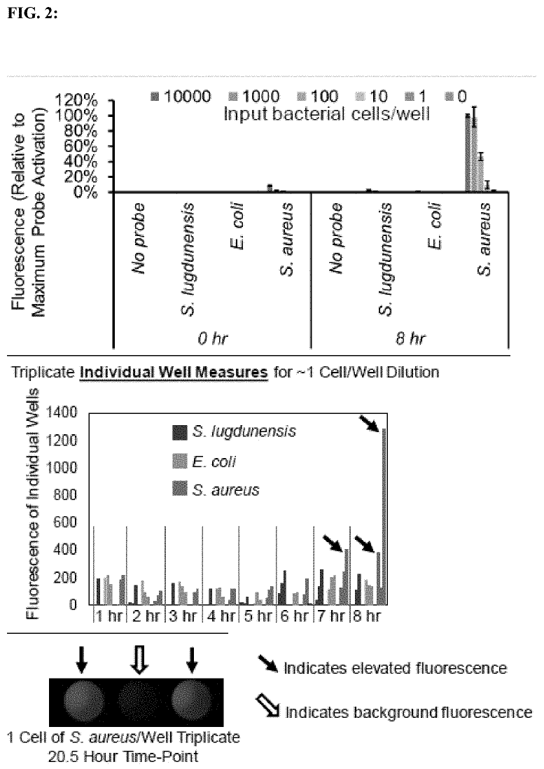

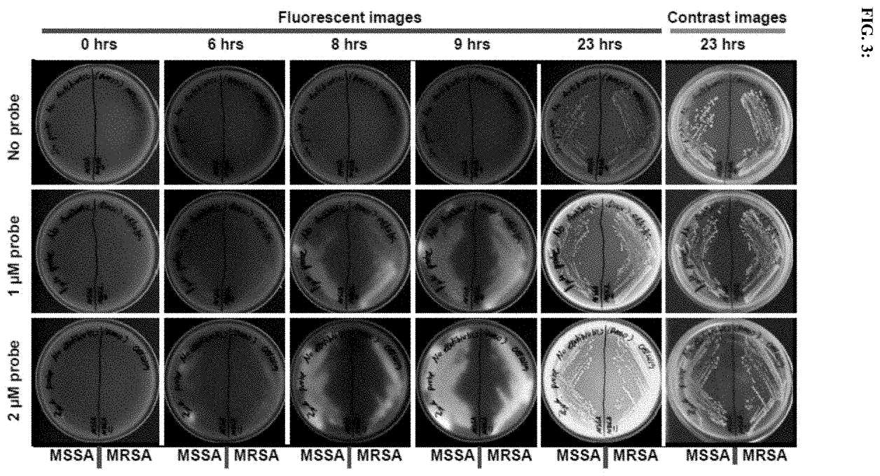

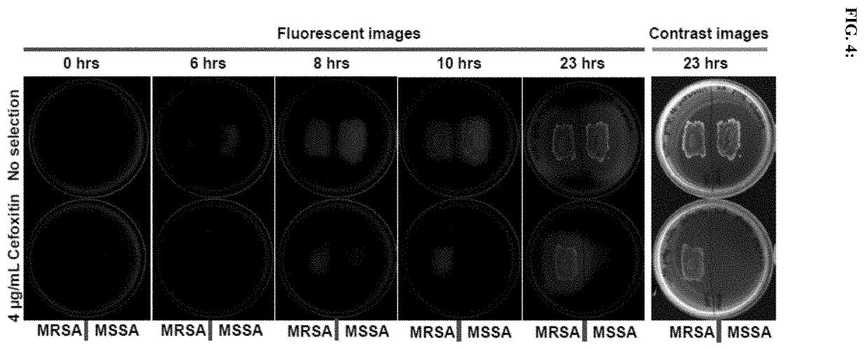

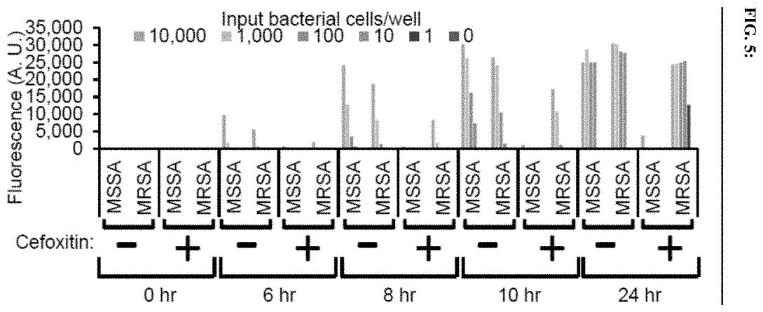

We also evaluated this S. aureus-responsive fluorogenic agar in the 96-well plate format. Low S. aureus loads produced a robust fluorescence signal within 8 hours (FIG. 2). The fluorogenic agar again exhibited specificity for S. aureus as neither E. coli nor S. lugdunensis yielded substantial fluorescence development within 8 hours. The fluorescence of the S. aureus 1 cell/well dilution was elevated in two of the 3 wells plated with this dilution at 8 hours (see FIG. 2, middle and lower panels; note: this elevation is less apparent in the upper panel of FIG. 2 due to the scale used for the graph). Dilution of the bacteria to ˜1 cell/well yields an occasional well with no cells due to the random distribution of bacteria. Indeed, close examination of the fluorescence of individual wells plated with this low concentration of S. aureus has revealed occasional wells that do not develop fluorescence, even after overnight incubations. Two of the 3 wells plated with the 1 cell/well S. aureus dilution developed elevated fluorescence after overnight incubation (FIG. 2, lower panel); these are the same two wells that showed elevated fluorescence at the 8-hour time point (see arrows in FIG. 2, middle and lower panels). These results, together with those of the streaked petri dishes (FIG. 1), support the conclusion that the methods described herein can detect very low bacterial loads of S. aureus within 8 hours, likely as low as 1 CFU. In addition to enabling rapid detection of Methicillin-sensitive Staphylococcus aureus (MSSA), this method was also effective for rapidly detecting Methicillin-resistant Staphylococcus aureus (MRSA) (FIGS. 3-5). MRSA detection was enabled by inclusion of cefoxitin in the agar in addition to the probe (i.e., an established means of isolating the growth of MRSA vs. MSSA) (FIGS. 4 & 5). As shown in FIGS. 4 & 5, MRSA can be specifically detected as early as 8 hours after inoculation with this approach. Based on these results, it is anticipated that this fluorogenic agar will enable MRSA screening of nasal swab specimens within 8 hours of collection. It is envisioned here that integration of this media into clinical workflows in an almost identical manner to that of chromogenic media that is currently used for routine MRSA screening will enable more rapid detection of MRSA. An additional application that could be enabled by the media is inexpensive and rapid identification of MSSA/MRSA in positive blood cultures. The simplicity, ease of use, and cost of this approach could make it an attractive alternative to more expensive and technically demanding molecular methods, particularly in low resource settings in developed countries and in low- and middle-income countries.

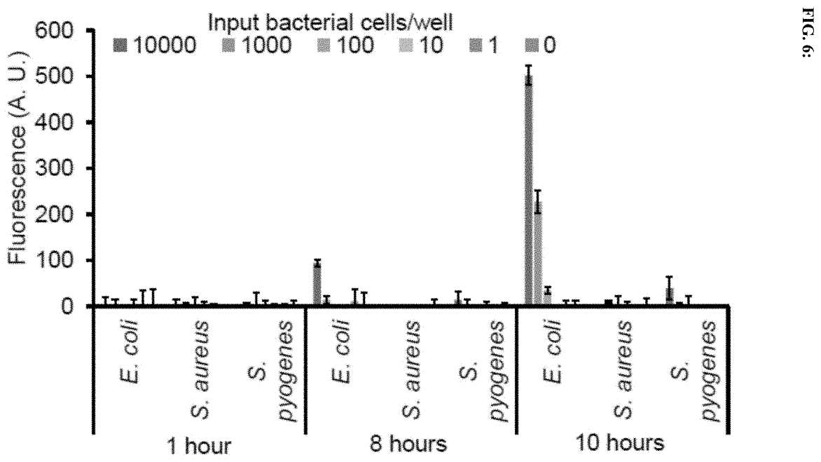

As all living organisms express nucleases, nuclease-based detection of additional bacterial species can in theory also be accomplished with the approach that is demonstrated for S. aureus. E. coli is another high impact bacterial pathogen and a member of the Enterobacteriaceae family which includes many additional, related pathogenic bacterial species. We developed a means of detecting E. coli and other members of the Enterobacteriaceae with a fluorogenic oligonucleotide probe that is efficiently digested by endonuclease I, a nuclease that is well conserved in this family. Here, we developed an agar media formulation, which when combined with an endonuclease I-responsive probe, enables the specific detection of E. coli (vs. S. aureus and S. pyogenes) within 8-10 hours of incubation (FIG. 6).

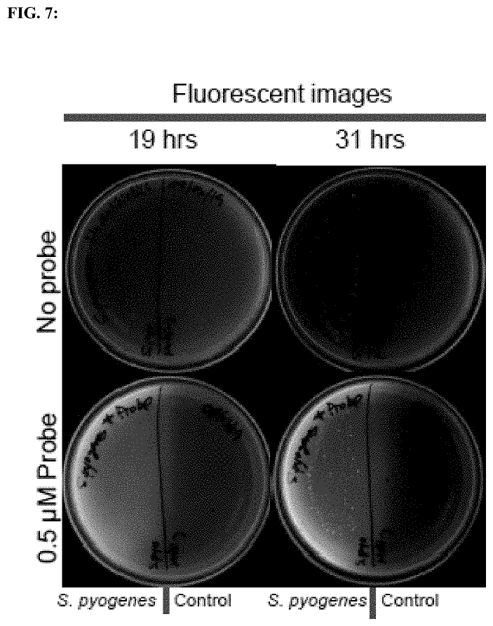

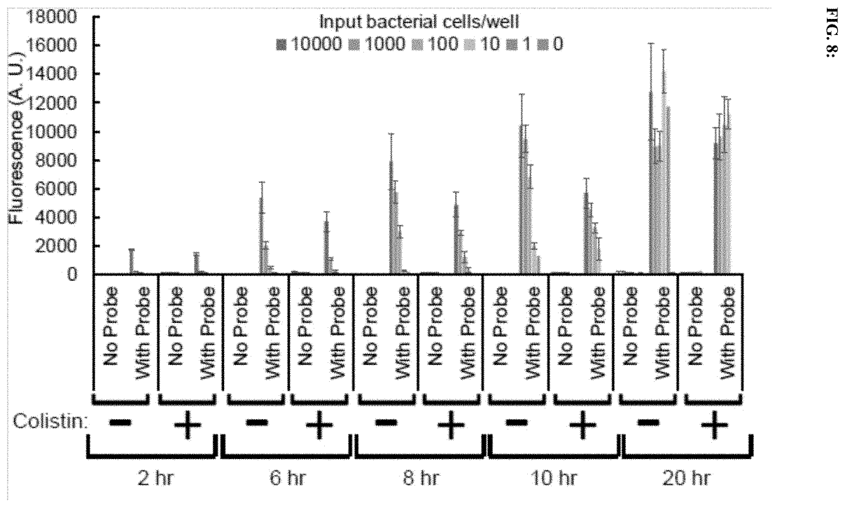

S. pyogenes, a high impact gram positive bacterial pathogen, can also be detected with nuclease-activatable agar, as shown in FIG. 7 (with bacteria streaked on agar plates) and FIG. 8 (with bacteria added to agar in a 96-well plate). In FIG. 7, the fluorescence of a plate with probe embedded is elevated only on the side where S. pyogenes is streaked; the fluorescence of a control plate that does not include probe is lower as is the side of the plate with probe embedded that is not streaked with bacteria. The experiment in FIG. 8 demonstrates the quantitative detection of S. pyogenes in the 96-well plate format, with signal developing in a bacterial load-dependent manner over the 20-hour time-course of the experiment. In addition, this experiment demonstrates the feasibility of combining the probe-based detection with colistin, an antibiotic that will prevent growth of most gram-negative bacterial species. While the nuclease probe used here is responsive to nucleases of some gram-negative bacterial species, such as E. coli, the inclusion of colistin provides a simple and well-established means of eliminating the growth of these species and thus enables specific detection of S. pyogenes.

### Example 3: Advantages of Using Fluorogenic Agar

MSSA/MRSA screening of nasal swabs is widely used to determine whether patients are colonized with these organisms prior to surgical procedures.

Detection of E. coli and related bacterial species is routinely carried out in the course of diagnosing urinary tract infections. Combination of probes for Enterobacteriaceae (E. coli is a member of this bacterial family) with antibiotics that would select for carbapenem resistant organisms could enable specific detection of carbapenem resistant Enterobacteriaceae (CRE). We have previously demonstrated that various members of the Enterobacteriaceae family respond to the probe we use here to detect E. coli (e.g., see Flenker, Katie S., et al. “Rapid detection of urinary tract infections via bacterial nuclease activity.” Molecular Therapy 25.6 (2017): 1353-1362). The functionality of this probe in detecting E. coli in the context of agar media is thus expected to be extendable to most of the Enterobacteriaceae and can enable detection of CRE. CRE screening of various samples such as perianal swabs is routine in many healthcare settings.

Rapid detection of MSSA/MRSA and CRE via fluorogenic agar could also enable rapid identification of these pathogens in positive blood cultures. This fluorogenic agar method would be especially useful for positive blood cultures in low resource settings due to the low costs of these plates.

S. pyogenes (group A Strep) often causes strep throat and is frequently screened for in throat swabs. Expensive ($30-$50) molecular tests can be used to determine the presence of this species in ˜2 hours. Less expensive, culture-based methods, such as chromogenic agars are often used, despite their much longer turnaround times of 18-24 hours. A simple method with a comparably low cost, but with a faster turnaround time could be an attractive alternative to the current culture-based methods.

### EQUIVALENTS

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth and as follows in the scope of the appended claims.

Those skilled in the art will recognize, or be able to ascertain, using no more than routine experimentation, numerous equivalents to the specific embodiments described specifically herein. Such equivalents are intended to be encompassed in the scope of the following claims.

### INCORPORATION BY REFERENCE

All patents and publications referenced herein are hereby incorporated by reference in their entireties.

What is claimed is:

1. A method for detecting a microorganism in a sample, comprising:
providing a culture substrate to support growth of a microorganism of interest, wherein the culture substrate comprises one or more antimicrobial agents and a nucleic acid probe that emits a fluorescent signal upon endonuclease cleavage;
contacting the culture substrate with the sample, and incubating the culture substrate for a period of time to allow growth of the microorganism of interest; and
detecting the presence, absence, or level of fluorescent signal in the culture substrate or a portion thereof, wherein detection of a fluorescent signal that is greater than the background or control indicates the presence of the microorganism of interest.

2. The method of claim 1, wherein the presence, absence, or level of the fluorescent signal is detected within about 15 hours of incubation.

3. (canceled)

4. (canceled)

5. The method of claim 1, wherein the culture substrate comprises an agar-containing growth medium.

6. The method of claim 5, wherein the fluorescent signal is detected using a spectrofluorometer, a microplate reader, a fluorescence microscope, a fluorescence scanner, a flow cytometer, a fluorescence gel imaging device, a combination of a transilluminator, a light filter and a camera, or other fluorescence imaging device.

7. (canceled)

8. The method of claim 1, wherein the nucleic acid probe comprises:
an oligonucleotide of 2-30 nucleotides in length comprising at least one endonuclease cleavage site;
at least one fluorescence quencher linked to the oligonucleotide;
at least one fluorophore linked to the oligonucleotide; and
wherein the fluorophore and fluorescence quencher flank the endonuclease cleavage site.

9. The method of claim 8, wherein cleavage by the microorganism's endonuclease separates the fluorophore from the fluorescence quencher allowing generation of the fluorescent signal.

10. The method of claim 8, wherein the oligonucleotide comprises one or more pyrimidines and at least one of the pyrimidines of the oligonucleotide is chemically modified.

11. (canceled)

12. (canceled)

13. The method of claim 8, wherein the oligonucleotide comprises one or more purines and at least one of the purines of the oligonucleotide is chemically modified.

14. (canceled)

15. (canceled)

16. The method of claim 8, wherein the probe comprises two oligonucleotides that are completely self-complementary yielding a double-stranded nucleic acid.

17. The method of claim 8, wherein the oligonucleotide comprises both RNA and DNA.

18. (canceled)

19. (canceled)

20. (canceled)

21. The method of claim 8, wherein the microorganism of interest is S. aureus.

22. The method of claim 21, wherein the antimicrobial agent is cefoxitin, and wherein fluorescent signal is indicative of the presence of MRSA.

23. (canceled)

24. (canceled)

25. (canceled)

26. The method of claim 8, wherein the culture substrate comprises colistin.

27. (canceled)

28. The method of claim 8, wherein the fluorescent probe is an endonuclease I-responsive probe, and optionally comprises an oligonucleotide sequence selected from CTACGTAG, fUfUfUfAfUfGfCfAfUfAfAfA, and fCfUfAfCfGfUfAfG, wherein f indicates 2′ Fluoro.

29. (canceled)

30. The method of claim 1, wherein the microorganism of interest secretes an endonuclease.

31. (canceled)

32. The method of claim 30, wherein the microorganism of interest is selected from Staphylococcus aureus, Staphylococcus epidermidis, Staphylococcus lugdunensis, Staphylococcus saprophyticus, Streptococcus pyogenes, Streptococcus agalactiae, Streptococcus pneumoniae, Streptococcus mutans, Listeria monocytogenes, Corynebacterium diphtherias, Bordetella pertussis, Clostridium difficile, Clostridium perfringens, Clostridium botulinum, Enterobacter cloacae, Citrobacter freundii, Borrelia burgdorferi, Treponema pallidum, Bacillus anthracis, Bacillus cereus, Enterococcus faecalis, Enterococcus faecium, vancomycin-resistant enterococci, Pseudomonas aeruginosa, Acinetobacter baumannii, Yersinia pestis, Yersinia pseudotuberculosis, Yersinia enterocolitica, Klebsiella pneumoniae, Vibrio cholerae, Salmonella enterica, Salmonella typhi, Escherichia coli, Serratia marcescens, Proteus mirabilis, Enterobacteriaceae, carbapenem-resistant Enterobacteriaceae, Candida auris, Aspergillus spp., Neisseria gonorrhoeae, Neisseria meningitidis, Mycobacterium tuberculosis, Haemophilus influenzae, Legionella pneumophila, Francisella tularensis, Bacteroides fragilis, Brucella abortus, Mycoplasma fermentans, Mycoplasma pneumonia, Mycoplasma bovis, Mycoplasma genitalium, Chlamydia trachomatis, Methicillin-resistant Staphylococcus aureus (MRSA), and Methicillin-sensitive Staphylococcus aureus (MSSA).

33. (canceled)

34. (canceled)

35. The method of claim 1, wherein the sample is a biological sample, wherein the biological sample is selected from a blood sample, a plasma sample, a urinary sample, a nasal swab, a throat swab, a stool sample, a saliva sample, a tissue sample, a hair sample, a skin sample, a skin swab, a bronchial aspirate sample, a bronchial lavage sample, a perianal swab sample, a synovial fluid sample, a cerebrospinal fluid sample, a blood culture sample, a bacterial culture isolate sample, a urine culture sample, and a surgical biopsy sample.

36. The method of claim 8, wherein the at least one fluorophore is selected from the fluorophores listed in Table 2.

37. (canceled)

38. The method of claim 8, wherein the at least one fluorophore has an emission in the near infra-red range.

39. (canceled)

40. (canceled)

41. (canceled)

42. The method of claim 1, further comprising a step of measuring a control fluorescent signal of one or more of:
a) a portion of the culture substrate which has not been contacted by the sample; or
b) a control culture substrate that does not include the nucleic acid probe.

43. The method of claim 42, further comprising a step of comparing the control fluorescent signal with the fluorescent signal.

44. The method of claim 1, wherein the antimicrobial agent is selected from the group consisting of Ceftriaxone, Cefepime, Vabomere, Avycaz, Trimethoprim and Sulfamethoxazole, Streptomycin, Fosfomycin, Ciprofloxacin, Azithromycin, Amoxicillin, Beta-lactams, Methicillin, Oxacillin, Cefoxitin, Penams, Cephams, Monobactams, Carbapenems, Meropenem, Ertapenem, Imipenem, Cephalosporins, Cephamycins, Beta-lactamase inhibitors, Colistin, Penicillin, Tetracycline, Erythromycin, Gentamicin, Vancomycin, Ceftazidime, Levofloxacin, Linezolid, Daptomycin, Ceftaroline, Amphotericin B, Candicidin, Filipin, Hamycin, Natamycin, Nystatin, Rimocidin, Azoles, Imidazoles, Bifonazole, Butoconazole, Clotrimazole, Econazole, Fenticonazole, Isoconazole, Ketoconazole, Luliconazole, Miconazole, Omoconazole, Oxiconazole, Sertaconazole, Sulconazole, Tioconazole Triazoles, Albaconazole, Efinaconazole, Epoxiconazole, Fluconazole, Isavuconazole, Itraconazole, Posaconazole, Propiconazole, Ravuconazole, Terconazole, Voriconazole, Thiazoles, Abafungim, Allylamines, Amorolfin, Butenafine, Naftifine, Terbinafine, Echinocandins, Anidulafungin, Caspofungin, and Micafungin.

45. The method of claim 1, further comprising the step of illuminating the culture substrate with a light suitable for absorption by the nucleic acid probe and detecting the emitted fluorescent signal.

46. (canceled)

47. A nuclease-activatable culture substrate for detecting a microorganism of interest comprising:
an agar-containing growth medium;
one or more antimicrobial agents; and
a nucleic acid probe that emits a fluorescent signal upon endonuclease cleavage, wherein the nuclease-activatable culture substrate is capable of supporting growth of the antimicrobial agent-resistant microorganisms.

48-69. (canceled)


SEQUENCE LISTING (6 sequences)

SEQ ID NO: 1 - RNA, 8 nt, Artificial Sequence
  Synthetic Sequence
  misc_feature (1)..(8): Each base pair in this sequence is a 2′-fluoro modified nucleotide. Please see Table 1 of the Specification.
cuacguag

SEQ ID NO: 2 - RNA, 8 nt, Artificial Sequence
  Synthetic Sequence
  misc_feature (1)..(8): Please see Table 1 of the Specification for the modified nucleotide description for this sequence. Each nucletotide is either a 2′-O-methyl modified nucleotide, or a RNA nucleotide.
cuacguag

SEQ ID NO: 3 - RNA, 8 nt, Artificial Sequence
  Synthetic Sequence
  misc_feature (1)..(8): Each nucleotide in this sequence is 2′-O-methyl modified nucleotide. Please see Table 1 of the Specification for the modified nucleotide description for this sequence.
cuacguag

SEQ ID NO: 4 - DNA, 8 bp, Artificial Sequence
  Synthetic Sequence
ctacgtag

SEQ ID NO: 5 - RNA, 12 nt, Artificial Sequence
  Synthetic Sequence
  misc_feature (1)..(12): Each base pair in this sequence is a 2′-fluoro modified nucleotide. Please see Table 1 of the Specification for the modified nucleotide description for this sequence.
uuuaugcauaaa

SEQ ID NO: 6 - RNA, 11 nt, Artificial Sequence
  Synthetic Sequence
  misc_feature (1)..(11): Each U nucleotide in this sequence is a 2′-O- methyl modified RNA nucleotide; the n nucleotides are deoxythymidine. Please see Table 1 of the Specification for the modified nucleotide description for this sequence.
unnuuuuuuuu